(19) United States
(12) Patent Application Publication
(10) Pub. No.: US 2026/0069282 A1
(43) Pub. Date: Mar. 12, 2026

(54) HEMOSTATIC POWDER DELIVERY DEVICES AND METHODS OF USE

(71) Applicants: Boston Scientific Scimed, Inc., Maple Grove, MN (US); Boston Scientific Medical Device Limited, Galway (IE)

(72) Inventors: Nachiket GOLE, Pune (IN); Rajivkumar SINGH, Pune (IN); Sharath Kumar G, Kanakapura (IN); Deepak Kumar SHARMA, Gurgaon (IN); Rosangel Aimee RAMOS ESPINOZA, Boston, MA (US)

(73) Assignees: Boston Scientific Scimed, Inc., Maple Grove, MN (US); Boston Scientific Medical Device Limited, Galway (IE)

(21) Appl. No.: 19/325,665

(22) Filed: Sep. 11, 2025

Related U.S. Application Data
(60) Provisional application No. 63/693,804, filed on Sep. 12, 2024.

(51) Int. Cl. A61B 17/12; A61B 17/00

(52) U.S. Cl. CPC A61B 17/12181; A61B 2017/0034; A61B 2017/00398; A61B 2017/00548; A61B 2017/00818; A61B 2017/12004; A61B 2017/1205

(57) ABSTRACT

A device for delivering an agent includes a valve assembly including a cavity for storing the agent and an agitation mechanism movably disposed inside the cavity. An inlet conduit fluidly coupled to a fluid source and the valve assembly. An outlet conduit fluidly coupled to the valve assembly, wherein the inlet and outlet conduit are each in fluid communication with the cavity. The agitation mechanism is configured to agitate the agent within the cavity in response to the agitation mechanism moving inside the cavity. The valve assembly is configured to urge the agitation mechanism to move inside the cavity in response to receiving a fluid from the fluid source via the inlet conduit, combine the fluid with the agent within the cavity in response to the agitation mechanism moving inside the cavity, and deliver a combination of the agent and the fluid out of the cavity through the outlet conduit.

### CROSS-REFERENCE TO RELATED APPLICATIONS

The present application claims the benefit of priority to U.S. Provisional Application No. 63/693,804, filed on Sep. 12, 2024, which is incorporated herein by reference in its entirety.

### TECHNICAL FIELD

Various embodiments of the present disclosure relate generally to devices and methods for delivering agents. More specifically, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a delivery device inserted into the working channel of the endoscope. Agent delivery may be achieved, for example, through mechanical systems. Such systems, however, may require numerous steps or actuations to achieve delivery, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract. The current disclosure may solve one or more of these issues or other issues in the art.

### SUMMARY OF THE DISCLOSURE

Aspects of the disclosure relate to, among other things, systems, devices, and methods for a medical device including a valve assembly for delivering agents. More specifically, this disclosure relates to devices for delivery of powdered agents, such as hemostatic agents, via a valve assembly that includes a cavity for storing the hemostatic agent and an agitation mechanism that is movably disposed inside the cavity to facilitate delivery of the hemostatic agent out of the device. Each of the aspects disclosed herein may include one or more of the features described in connection with any of the other disclosed aspects.

According to an example, a device for delivering an agent includes a valve assembly including a cavity for storing the agent and at least one agitation mechanism movably disposed inside the cavity; an inlet conduit fluidly coupled to a fluid source and the valve assembly; and an outlet conduit fluidly coupled to the valve assembly, wherein the inlet conduit and the outlet conduit are each in fluid communication with the cavity; wherein the at least one agitation mechanism is configured to agitate the agent within the cavity in response to the at least one agitation mechanism moving inside the cavity; wherein the valve assembly is configured to urge the at least one agitation mechanism to move inside the cavity in response to receiving a fluid from the fluid source via the inlet conduit; combine the fluid with the agent within the cavity in response to the at least one agitation mechanism moving inside the cavity; and deliver a combination of the agent and the fluid out of the cavity through the outlet conduit.

Any of the devices described herein may include any of the following features. The fluid source includes a pressurized canister, and the fluid includes a pressurized gas. The cavity includes at least one measured dose of the agent. When combining the agent with the fluid within the cavity, the valve assembly is configured to direct the fluid towards the at least one agitation mechanism, thereby moving the at least one agitation mechanism within the cavity and agitating the agent. The valve assembly includes an outlet port coupled to the outlet conduit and a mesh coupled to the outlet port; wherein the mesh is configured to inhibit at least a portion of the agent from exiting the cavity via the outlet port, and the outlet port includes a funnel shape configured to guide the combination of the agent and the fluid towards the outlet conduit. The at least one agitation mechanism includes a ball that is configured to generate turbulence within the cavity to combine the fluid and the agent in response to the fluid from the inlet conduit urging the at least one agitation mechanism to move within the cavity. The valve assembly includes one or more interior surfaces defining the cavity; and a wire extending along at least one of the one or more interior surfaces, wherein the wire is configured to generate a magnetic pulse and the at least one agitation mechanism is magnetic; and wherein the wire is configured to magnetically couple with the at least one agitation mechanism when generating the magnetic pulse, thereby causing the at least one agitation mechanism to move within the cavity and relative to the one or more interior surfaces to agitate the agent. The valve assembly includes: one or more interior surfaces defining the cavity; and a membrane positioned adjacent to at least one of the one or more interior surfaces; wherein the membrane is configured to agitate the agent with the fluid received within the cavity in response to the membrane vibrating against the at least one of the one or more interior surfaces. The valve assembly includes a base including at least one magnetic mechanism disposed adjacent to the cavity, and the at least one agitation mechanism is magnetic; wherein the base is configured to move relative to the cavity and the at least one magnetic mechanism is configured to magnetically couple with the at least one agitation mechanism, thereby causing the at least one agitation mechanism to move within the cavity and agitate the agent. The at least one agitation mechanism is configured to create a vortex within the cavity in response to the at least one agitation mechanism moving therein, thereby agitating the agent with the fluid. The base includes a central body and a plurality of blades coupled to the central body, at least one of the plurality of blades includes the at least one magnetic mechanism; wherein the central body is configured to rotate the plurality of blades relative to the cavity, thereby causing the at least one magnetic mechanism to rotate the at least one agitation mechanism. The at least one magnetic mechanism is configured to generate a magnetic field that moves the at least one agitation mechanism within the cavity. The valve assembly includes an outer enclosure and an inner enclosure disposed inside the outer enclosure, wherein the inner enclosure includes one or more interior surfaces defining the cavity; and wherein the inner enclosure is configured to move relative to the outer enclosure, thereby causing the at least one agitation mechanism to move within the cavity to agitate the agent with the fluid. The one or more interior surfaces are magnetic and the at least one agitation mechanism is magnetic, such that the one or more interior surfaces are configured to magnetically couple with the at least one agitation mechanism. The one or more interior surfaces are configured to generate a magnetic field, thereby causing the at least one agitation mechanism to move within the cavity to agitate the agent in response to movement of the inner enclosure relative to the outer enclosure.

According to another example, a device for delivering an agent includes a valve assembly including a cavity and an agitation mechanism movably disposed inside the cavity; and a base including a magnetic mechanism movably disposed outside the cavity, wherein the magnetic mechanism is magnetically coupled to the agitation mechanism, and the base is configured to move relative to the valve assembly, thereby causing movement of the magnetic mechanism; wherein the agitation mechanism is configured to move within the cavity to agitate the agent stored therein in response to movement of the magnetic mechanism outside the cavity; and wherein the valve assembly is configured to mix the agent with a fluid received within the cavity and deliver a mixture of the agent and the fluid out of the cavity in response to movement of the agitation mechanism in the cavity.

Any of the devices described herein may include any of the following features. The agitation mechanism is magnetic, and the magnetic mechanism is configured to urge the agitation mechanism to move within the cavity in response to a corresponding movement of the base outside the cavity. The base includes a central body and a plurality of blades extending outwardly from the central body, wherein the magnetic mechanism is coupled to at least one of the plurality of blades. The plurality of blades are configured to rotate relative to the valve assembly, thereby rotating the magnetic mechanism relative to the cavity.

According to a further example, a device for delivering an agent includes a valve assembly including an outer enclosure, an inner enclosure including a cavity storing the agent, and an agitation mechanism, the inner enclosure is movably disposed inside the outer enclosure and the agitation mechanism is movably disposed inside the cavity; wherein the inner enclosure is magnetically coupled to the agitation mechanism, such that the inner enclosure is configured to move the agitation mechanism within the cavity in response to moving relative to the outer enclosure; and wherein the agitation mechanism is configured to agitate the agent stored inside the cavity in response to the inner enclosure moving within the outer enclosure.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device, according to one or more embodiments.

FIG. 2 shows a side view of an exemplary valve assembly of the delivery device of FIG. 1, according to one or more embodiments.

FIG. 3A shows a perspective view of the valve assembly of FIG. 2 in a first state, according to one or more embodiments.

FIG. 3B shows a cross-sectional side view of the valve assembly of FIG. 2 in a second state, according to one or more embodiments.

FIG. 4 shows a perspective view of another exemplary valve assembly of the delivery device of FIG. 1, according to one or more embodiments.

FIG. 5 shows a top view of a base of the valve assembly of FIG. 4, according to one or more embodiments.

FIG. 6 shows a perspective view of another exemplary valve assembly of the delivery device of FIG. 1, according to one or more embodiments.

FIG. 7 shows a perspective view of another exemplary valve assembly of the delivery device of FIG. 1, according to one or more embodiments.

FIG. 8 shows a perspective view of another exemplary valve assembly of the delivery device of FIG. 1, according to one or more embodiments.

### DETAILED DESCRIPTION OF EMBODIMENTS

Embodiments of this disclosure relate to dispensing devices having valve assemblies for selectively releasing a material (e.g., a powdered agent) to a site of a medical procedure. In other words, the valve assemblies may be configured and operable to move stagnant material and facilitate proper dispensing of the material. The valve assembly may include at least one agitation mechanism movably disposed inside a cavity of an enclosure, in order to combine the agent and a fluid. The cavity may store a predetermined amount of the agent, and may be in fluid communication with a pressurized fluid medium source (e.g., a gas canister). Upon release of a pressurized fluid into the cavity, the agent and the fluid may be combined due to the movement agitation mechanism within the cavity. In some embodiments, movement of the agitation mechanism may be caused by the pressurized fluid received within the cavity.

The agent may be in fluid communication with the pressurized fluid through an inlet conduit and/or opening formed by the valve assembly. Accordingly, when the agent is combined with pressurized fluid, the valve assembly delivers the combination of the agent and the pressurized fluid through an outlet conduit to a target treatment site during a medical procedure. Aspects of the dispensing device and valve assembly, such as the at least one agitation mechanism, may facilitate agitation and combination of the agent and the pressurized fluid prior to dispensing to prevent or minimize clogging of the dispensing device during delivery. The valve assembly may be configured and operable to move the agent that may be stored in a stagnant state and facilitate proper dispensing of the agent out of the dispensing device.

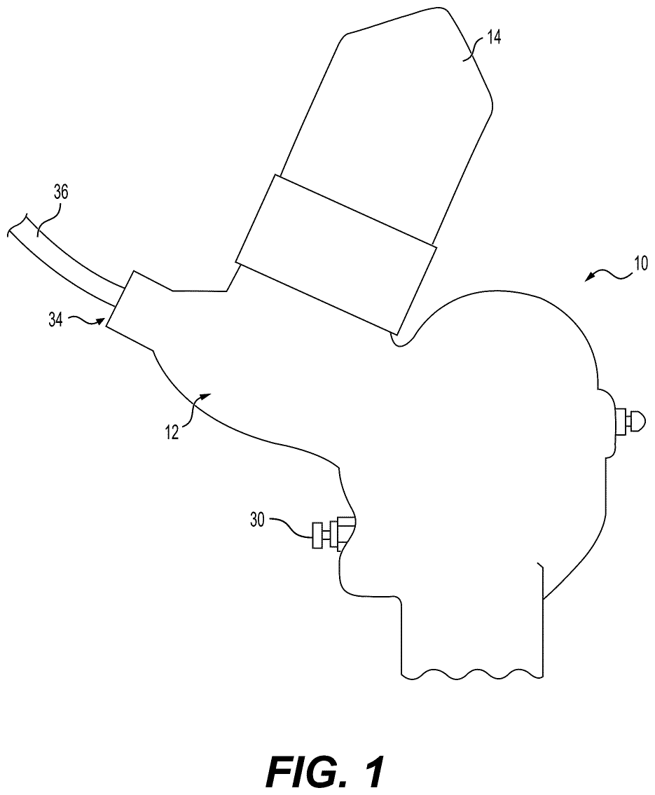

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. patent application Ser. No. 16/589,633, filed Oct. 1, 2019, published as U.S. Pat. No. 11,642,281 B1 on May 9, 2023, the disclosure of which is hereby incorporated by reference in its entirety, discloses features of exemplary delivery devices and systems. The features of this disclosure may be combined with any of the features described in the above-referenced patent. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source (e.g., gas canister) in fluid communication with delivery system 10. Actuation mechanism 30 may be selectively actuated (e.g., manually depressible) or otherwise moved or actuated to control delivery of a material (e.g., a powdered agent) and/or the pressurized fluid. The pressurized fluid alone, or a combination of a powdered agent and the pressurized fluid, may be delivered from an outlet 34 of handle body 12. Outlet 34 may be in fluid communication with a delivery conduit, for example a catheter 36 or another component for delivering the combination of agent and fluid to a desired location within a body lumen of a patient. Delivery system 10 may include a valve assembly for selectively controlling the release of the powdered agent and/or the pressurized fluid within enclosure 14.

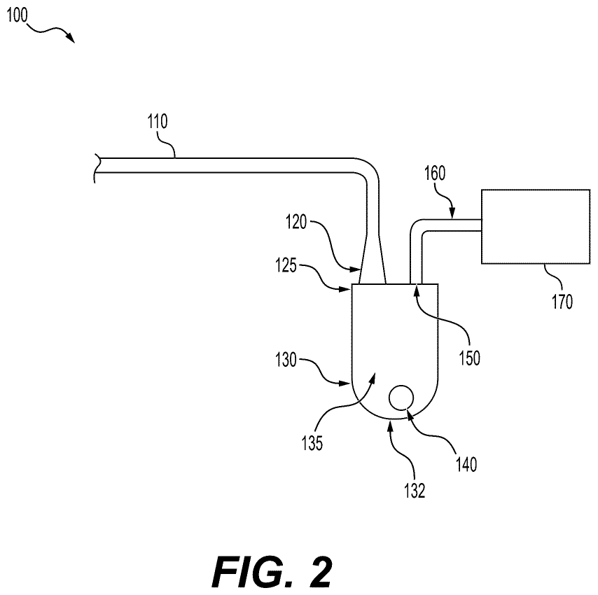

FIG. 2 shows an exemplary valve assembly 100, according to one or more embodiments. Valve assembly 100 may be part of delivery system 10 as discussed with reference to FIG. 1 above, such as disposed in enclosure 14. Valve assembly 100 may include an outlet conduit 110 in fluid communication with an outlet port 120, an enclosure 130 (e.g., powder chamber) in fluid communication with outlet port 120, an inlet port 150 in fluid communication with enclosure 130, and an inlet conduit 160 in fluid communication with inlet port 150. In other words, enclosure 130 is fluidly coupled to outlet conduit 110 via outlet port 120 and inlet conduit 160 via inlet port 150. Enclosure 130 may include a lid 125, with outlet port 120 and inlet port 150 defining openings disposed on lid 125. Enclosure 130, lid 125, and a bottom surface 132 of the enclosure 130 may collectively define a cavity 135 within enclosure 130 for storing a material (e.g., powdered agent).

Bottom surface 132 may have various suitable sizes, shapes, configurations, and/or surface finishes for facilitating storage and/or delivery of the powdered agent from cavity 135, such as upon receipt of the pressurized fluid in enclosure 130 as described in detail herein. In the example, bottom surface 132 may include a shape defining a hemispherical surface with a smooth finish. Enclosure 130 may include an agitation mechanism 140 movably disposed inside the cavity 135. Agitation mechanism 140 may be configured to move within enclosure 130 to agitate, move, stir, and/or mix the powdered agent stored inside cavity 135, such as in response to enclosure 130 receiving a pressurized fluid from inlet port 150 via inlet conduit 160. For example, agitation mechanism 140 may be configured to spin rapidly within cavity 135 due to receipt of the pressurized fluid in enclosure 130 from inlet port 150, thereby agitating and mixing the powdered agent with the pressurized fluid prior to delivering the mixture out of enclosure 130 via outlet port 120.

Still referring to FIG. 2, agitation mechanism 140 may include various suitable sizes, shapes, and/or cross-sectional configurations for generating maximum turbulence within cavity 135 to agitate and temporarily suspend the powdered agent stored therein. For example, agitation mechanism 140 may include a ball, a pin, a piston, a body, or other mechanism that is sized, shaped, and/or otherwise configured to agitate a powdered agent. It should be appreciated that agitation mechanism 140 may be formed of various suitable material, including but not limited to, plastic (e.g., polyethylene). In the example, agitation mechanism 140 may include a ball having a spherical and/or oblong shape. Valve assembly 100 may include a pressurized fluid source 170 (e.g., canister) in fluid communication with inlet conduit 160, and pressurized fluid source 170 may be configured to provide a pressurized fluid into cavity 135 through inlet port 150 via inlet conduit 160.

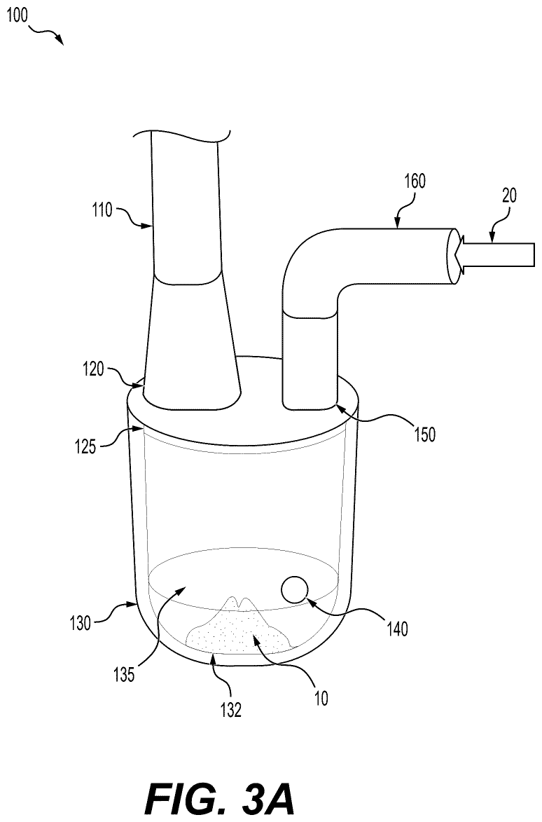

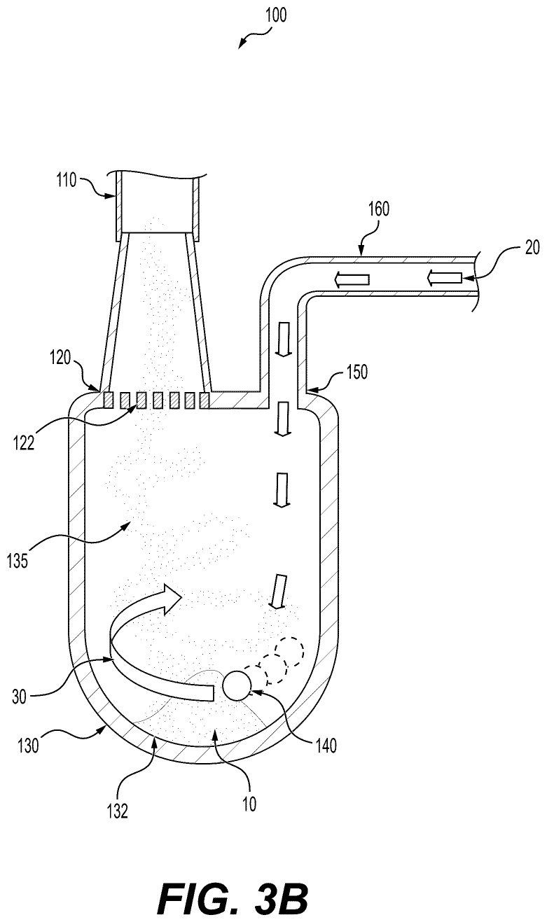

FIG. 3A shows valve assembly 100 in a first (non-fluidized) state with enclosure 130 storing a powdered agent 10 prior to receipt of a fluid flow 20 of pressurized fluid from pressurized fluid source 170 (see FIG. 2) via inlet conduit 160. Valve assembly 100 may include one or more measured doses of powdered agent 10 disposed within cavity 135. In some embodiments, valve assembly 100 may include a single measured dose of powdered agent 10. In other embodiments, valve assembly 100 may include multiple measured doses of powdered agent 10 for delivery to a subject (e.g., patient). Pressurized fluid source 170 (FIG. 2) may be configured to deliver fluid flow 20 of pressurized fluid into the inlet conduit 160 for receipt within enclosure 130 via inlet port 150. Activation of pressurized fluid source 170 may be in response to actuation of actuation mechanism 30 of delivery device 10 (FIG. 1).

Referring now to FIG. 3B, valve assembly 100 may be in a second (fluidized) state with enclosure 130 receiving fluid flow 20 of pressurized fluid from pressurized fluid source 170 (FIG. 2), thereby causing movement of agitation mechanism 140 within cavity 135. In other words, valve assembly 100 may be configured to, upon activation of actuation mechanism 30, receive fluid flow 20 of pressurized fluid from pressurized fluid source 170 at inlet port 150 via inlet conduit 160. In some embodiments, fluid flow 20 may continue to flow into cavity 135 upon continued actuation of actuation mechanism 30. In other embodiments, each actuation of actuation mechanism 30 may be configured to release a predefined volume of pressurized fluid to enclosure 130 via fluid flow 20.

A relative position and/or orientation of inlet port 150 on lid 125 relative to enclosure 130, and/or a pressurization level of the fluid flow 20 of pressurized fluid from the pressurized fluid source 170, or a combination thereof, may direct and/or force at least a portion of the fluid flow 20 towards bottom surface 132. In the example, inlet port 150 may be arranged opposite to, and facing downwards towards, bottom surface 132. With agitation mechanism 140 disposed inside cavity 135 adjacent to bottom surface 132, inlet port 150 may be configured to guide fluid flow 20 towards agitation mechanism 140, thereby causing the fluid flow 20 to encounter and urge agitation mechanism 140 to move inside cavity 135.

Agitation mechanism 140 may be configured to agitate and/or move powdered agent 10 within cavity 135 in response to agitation mechanism 140 moving inside enclosure 130. Fluid flow 20 and agitation mechanism 140 may collectively create a vortex 30 within cavity 135, thereby combining powdered agent 10 and the pressurized fluid received within enclosure 130. Vortex 30 may be created in response to fluid flow 20 being directed inside cavity 135 towards bottom surface 132 of enclosure 130. Vortex 30 may be configured to generate turbulence within cavity 135, thereby further urging agitation mechanism 140 to move inside cavity 135 to mix with powdered agent 10. Agitation mechanism 140 may be configured to mix powdered agent 10 and the pressurized fluid prior to dispensing the mixture out of enclosure 130 to help prevent and/or minimize clogging of outlet conduit 110 and/or catheter 36 (see FIG. 1) during delivery of powdered agent 10.

Still referring to FIG. 3B, agitation mechanism 140 may be configured to guide and/or urge the combination of powdered agent 10 and fluid flow 20 of pressurized fluid out of cavity 135 of enclosure 130 towards outlet port 120. Outlet port 120 may be configured to deliver the mixture of powdered agent 10 and fluid flow 20 received from cavity 135 out through outlet conduit 110 and towards catheter 36 (FIG. 1). In some embodiments, outlet port 120 may be sized, shaped, and/or otherwise configured to define a funnel having a tapered profile between lid 125 and an end of outlet conduit 110. The funnel of outlet port 120 may be configured to guide the combination of powdered agent 10 and fluid flow 20 of pressurized fluid towards outlet conduit 110 for delivery.

In some embodiments, valve assembly 100 may include an outlet mesh 122 coupled to lid 125 at outlet port 120. Outlet mesh 122 may be configured to prevent and/or minimize clogging of outlet port 120, outlet conduit 110, and/or catheter 36 (FIG. 1) during delivery of the mixture of powdered agent 10 and pressurized fluid from delivery device 10. Outlet mesh 122 may include a porous structure defining one or more (e.g., a plurality) pores and/or openings that are each sized, shaped, and/or otherwise configured to permit particles of powdered agent 10 that do not exceed a maximum particulate size from extending through outlet mesh 122 and entering outlet port 120. In other words, the plurality of pores and/or openings of outlet mesh 122 may be configured to inhibit particles of powdered agent 10 that are greater than the maximum particulate size from exiting cavity 135 of enclosure 130 and entering into outlet port 120, until agitation mechanism 140 further agitates the particles to a relatively smaller cross-sectional dimension.

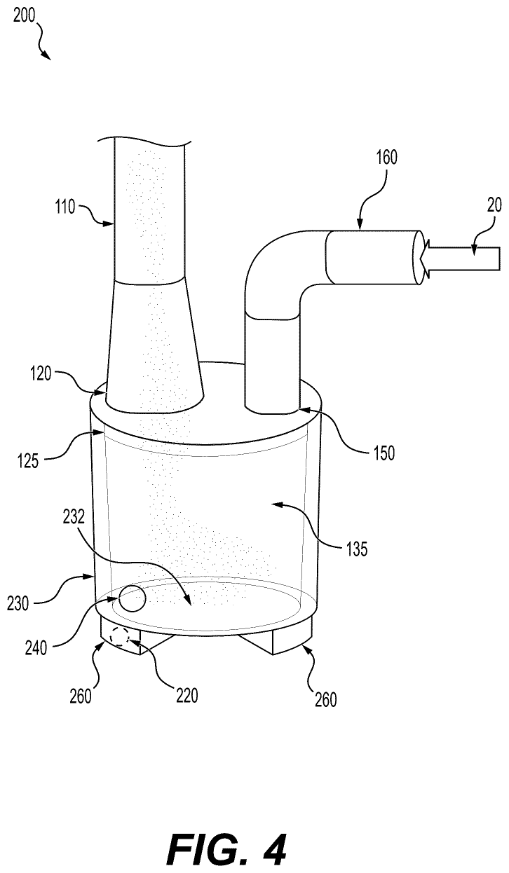

FIG. 4 shows another exemplary valve assembly 200, according to one or more embodiments. Valve assembly 200 may be configured and operable similar to valve assembly 100 shown and described above except as otherwise described herein. As such, like reference numerals are used to identify similar components. It should be appreciated that valve assembly 200 may be integrated in delivery device 10 (FIG. 1) in a substantially similar manner as valve assembly 100 described above. Valve assembly 200 may include an enclosure 230 that is in fluid communication with inlet conduit 160 via inlet port 150 on lid 125, and outlet conduit 110 via outlet port 120 on lid 125. Enclosure 230 may include a bottom surface 232 that at least partially defines cavity 135 with lid 125. In the example, bottom surface 232 may have a flat and/or planar configuration for receiving and storing powdered agent 10 thereon.

Enclosure 230 may include an agitation mechanism 240 disposed inside cavity 135. Agitation mechanism 240 may be configured to agitate powdered agent 10 in response to cavity 135 receiving fluid flow 20 of pressurized fluid at inlet port 150 via inlet conduit 160. Agitation mechanism 240 may include various suitable sizes, shapes, and/or cross-sectional configurations. For example, agitation mechanism 240 may include a ball, a pin, a piston, a body, or other mechanism that is sized, shaped, and/or otherwise configured to agitate powdered agent 10. In the example, agitation mechanism 240 may include a ball that is magnetic and has a spherical shape. Valve assembly 200 may include a base 210 movably coupled to enclosure 230, such as external to and adjacent to bottom surface 232. Base 210 may be configured to move relative to bottom surface 232 in one or more directions as described herein.

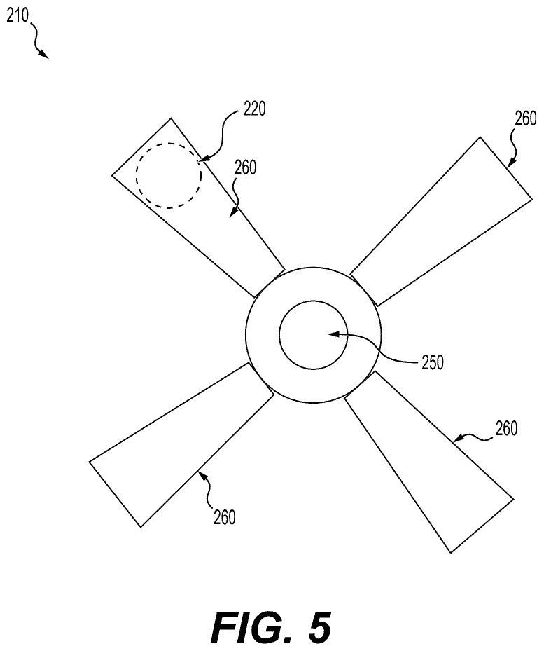

Referring now to FIG. 5, base 210 of valve assembly 200 may include a central body 250, one or more blades 260 coupled to and extending radially outwards from central body 250, and a magnetic mechanism 220 coupled to at least one of the one or more blades 260. In some embodiments, base 210 may include a plurality of blades 260. In the example, base 210 may include four blades 260 that extend about central body 250 in an annular array. Magnetic mechanism 220 may include a ball, a plate, a panel, or other mechanism that is magnetic and/or otherwise configured to magnetically couple with agitation mechanism 240 stored inside cavity 135. It should be appreciated that the one or more blades 260, and magnetic mechanism 220 coupled thereto, may be configured to move (e.g., rotate) relative to bottom surface 232 in response to a corresponding movement (e.g., rotation) of central body 250 relative to enclosure 230.

Valve assembly 200 may include a power source (not shown) configured to move central body 250 of base 210 relative to enclosure 230, thereby providing a corresponding movement of blades 260 and magnetic mechanism 220. In some embodiments, the power source may include an electrical motor. For example, base 210 may be configured to move (e.g. rotate) relative to a central axis of central body 250, such that blades 260 extending radially outwards from central body 250 may move (e.g. rotate) about the central axis defined by central body 250. Base 210 of valve assembly 200 may be disposed relatively below enclosure 230 with magnetic mechanism 220 positioned adjacent to and/or against an exterior wall of enclosure 230 defining bottom surface 232. Magnetic mechanism 220 may be configured to magnetically couple with agitation mechanism 240 such that movement of base 210 outside of enclosure 230 may cause a corresponding movement of agitation mechanism 240 inside enclosure 230 to facilitate agitation and delivery of powdered agent 10 from cavity 135 towards outlet port 120.

Still referring to FIG. 5, blades 260 and magnetic mechanism 220 may be configured to simultaneously rotate with central body 250 of base 210 relative to enclosure 230, thereby causing agitation mechanism 240 within cavity 135 to agitate powdered agent 10 and the pressurized fluid in enclosure 230 due to the magnetic coupling of agitation mechanism 240 with magnetic mechanism 220. Stated differently, with agitation mechanism 240 magnetically coupled to magnetic mechanism 220, agitation mechanism 240 may be configured to move (e.g. rotate) within enclosure 230 in response to a corresponding movement (e.g., rotation) of magnetic mechanism 220 external to enclosure 230. Agitation mechanism 240 may be configured to agitate and/or mix the powdered agent 10 and pressurized fluid within cavity 135 in response to agitation mechanism 240 moving inside enclosure 230 to prevent and/or minimize clogging of outlet conduit 110 and/or catheter 36 (FIG. 1) during delivery of powdered agent 10. In other embodiments, central body 250 may be fixed relative to enclosure 230 such that blades 260 and magnetic mechanism 220 may be configured to rotate relative to central body 250.

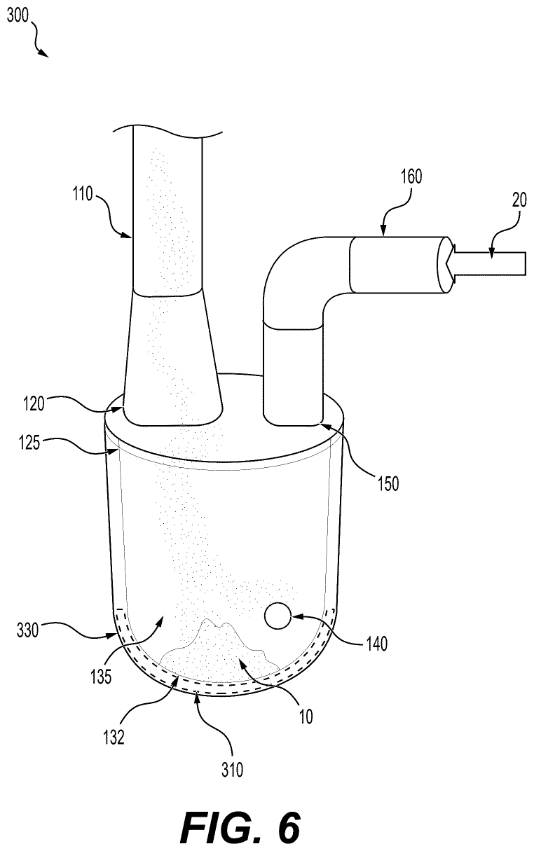

FIG. 6 shows another exemplary valve assembly 300, according to one or more embodiments. Valve assembly 300 may be configured and operable similar to valve assembly 100 shown and described above except as otherwise described herein. As such, like reference numerals are used to identify similar components. It should be appreciated that valve assembly 300 may be integrated in delivery device 10 (FIG. 1) in a substantially similar manner as valve assembly 100 described above. Valve assembly 300 may include an enclosure 330 that is in fluid communication with inlet conduit 160 via inlet port 150 on lid 125, and outlet conduit 110 via outlet port 120 on lid 125. Enclosure 330 may include bottom surface 132 that at least partially defines cavity 135 with lid 125.

Valve assembly 300 may include a membrane 310 positioned adjacent to bottom surface 132 of enclosure 330. In some embodiments, membrane 310 may be coupled to, disposed on, and/or otherwise in contact with bottom surface 132. In other embodiments, membrane 310 may be coupled to, disposed on, and/or otherwise in contact with one or more other surfaces and/or walls of enclosure 330 without departing from a scope of this disclosure. In the example, membrane 310 may be attached to enclosure 330 along an interior wall defining bottom surface 132.

Still referring to FIG. 6, membrane 310 may be configured and operable to generate a pulse, a vibration, and/or other movement that may be transferrable to bottom surface 132 due to membrane 310 being in close proximity and/or physical contact with bottom surface 132. As such, membrane 310 may be configured to cause a corresponding movement of bottom surface 132 upon activation of membrane 310. In some embodiments, membrane 310 may include a sound membrane that is configured to create sound pulses or vibrations at various suitable wavelengths and/or frequencies to produce a corresponding pulse or vibration of bottom surface 132 at said wavelength and/or frequency. Membrane 310 may be configured to manipulate and/or move powdered agent 10 stored along bottom surface 132 upon activation. In other embodiments, membrane 310 may be configured to manipulate and/or move agitation mechanism 140 within cavity 135 in response to moving and/or vibrating bottom surface 132 over which agitation mechanism 140 is disposed.

Valve assembly 300 may include a power source (not shown) configured to activate membrane 310 against bottom surface 132 of enclosure 330, thereby moving and/or vibrating agitation mechanism 140 inside enclosure 330. In some embodiments, the power source may include an electrical motor. Agitation mechanism 140 may be configured to agitate powdered agent 10 in response to the movement and/or vibration of bottom surface 132 by membrane 310, and combine the mixture of powdered agent 10 and pressurized fluid within cavity 135 prior to delivery out of enclosure 330 via outlet port 120. Agitation mechanism 140 may be configured to agitate and/or mix powdered agent 10 and pressurized fluid within cavity 135 in response to agitation mechanism 140 moving inside enclosure 330 to prevent and/or minimize clogging of outlet conduit 110 and/or catheter 36 (FIG. 1) during delivery of powdered agent 10.

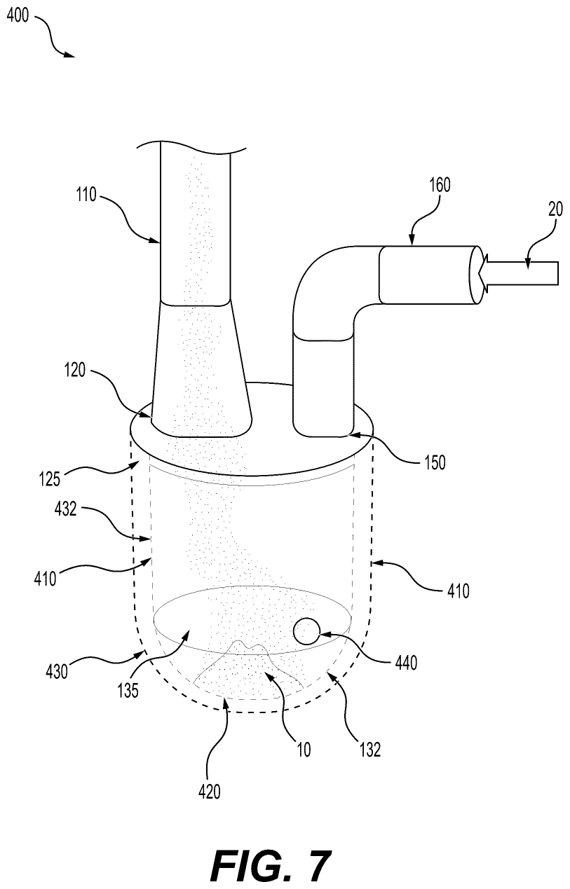

FIG. 7 shows another exemplary valve assembly 400, according to one or more embodiments. Valve assembly 400 may be configured and operable similar to valve assembly 100 shown and described above except as otherwise described herein. As such, like reference numerals are used to identify similar components. It should be appreciated that valve assembly 400 may be integrated in delivery device 10 (FIG. 1) in a substantially similar manner as valve assembly 100 described above. Valve assembly 400 may include an enclosure 430 that is in fluid communication with inlet conduit 160 via inlet port 150 on lid 125, and outlet conduit 110 via outlet port 120 on lid 125. Enclosure 430 may include bottom surface 132 that at least partially defines cavity 135 with lid 125. Enclosure 430 may include a first (side) magnetic wiring 410 and a second (bottom) magnetic wiring 420 disposed over one or more surfaces and/or walls of enclosure 430. For example, side magnetic wiring 410 may be disposed on and/or coupled to a sidewall 432 of enclosure 430, and bottom magnetic wiring 420 may be disposed on and/or coupled to bottom surface 132 of enclosure 430.

Although a pair of magnetic wiring 410, 420 is shown and described herein as being positioned on side and bottom surfaces and/or walls of enclosure 430, it should be appreciated that valve assembly 400 may include additional and/or fewer magnetic wirings, and at various other suitable locations relative to enclosure 430, without departing from a scope of this disclosure. For example, one or more of magnetic wiring 410, 420 may be positioned internal and/or external to cavity 135 of enclosure 430. In some embodiments, a position and/or an arrangement of magnetic wiring 410, 420 relative to enclosure 430 may be securely fixed. In other embodiments, a position and/or an arrangement of magnetic wiring 410, 420 relative to enclosure 430 may be selectively adjustable.

Still referring to FIG. 7, valve assembly 400 may include an agitation mechanism 440 disposed inside cavity 135 of enclosure 430. Agitation mechanism 440 may include various suitable sizes, shapes, and/or cross-sectional configurations. For example, agitation mechanism 440 may include a ball, a pin, a piston, a body, or other mechanism that is sized, shaped, and/or otherwise configured to agitate powdered agent 10. Agitation mechanism 440 may be positioned adjacent to one or more of sidewall 432 and bottom surface 132. Agitation mechanism 440 may be magnetic and/or include magnetic properties, such that agitation mechanism 440 may be configured to interact with side magnetic wiring 410 and bottom magnetic wiring 420 while disposed within enclosure 430.

Side magnetic wiring 410 and bottom magnetic wiring 420 may each be configured to independently and/or collectively create a magnetic field about cavity 135 of enclosure 430, such as to cause a corresponding movement of agitation mechanism 440 upon activation of magnetic wiring 410, 420. For example, magnetic wiring 410, 420 may be communicatively coupled to a power source (not shown) that is configured to activate each of magnetic wiring 410, 420. In some embodiments, the power source may include an electrical motor. In this instance, magnetic wiring 410, 420 may be configured and operable to magnetically interact with agitation mechanism 440, thereby causing agitation mechanism 440 to move within cavity 135. Agitation mechanism 440 may be configured to agitate powdered agent 10 and combine the mixture of powdered agent 10 and pressurized fluid within cavity 135 in response to agitation mechanism 240 moving inside enclosure 430 to prevent and/or minimize clogging of outlet conduit 110 and/or catheter 36 (see FIG. 1) during delivery of powdered agent 10.

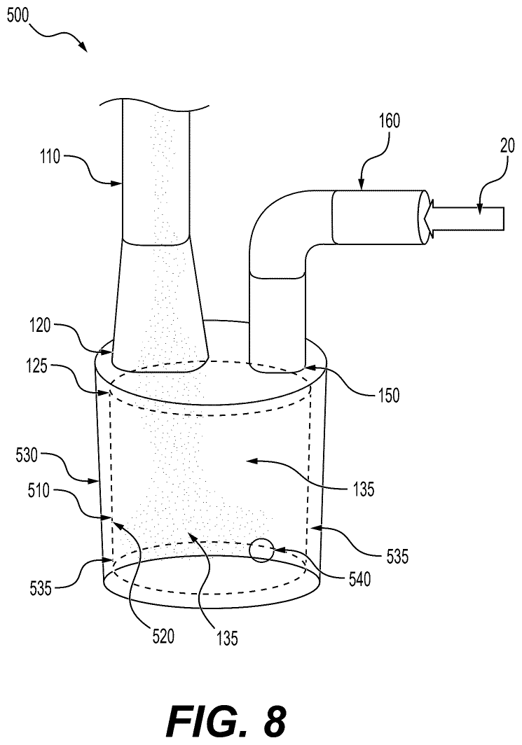

FIG. 8 shows another exemplary valve assembly 500, according to one or more embodiments. Valve assembly 500 may be configured and operable similar to valve assembly 100 shown and described above except as otherwise described herein. As such, like reference numerals are used to identify similar components. It should be appreciated that valve assembly 500 may be integrated in delivery device 10 (FIG. 1) in a substantially similar manner as valve assembly 100 described above. Valve assembly 500 may include an enclosure 530 that is in fluid communication with inlet conduit 160 via inlet port 150 on lid 125, and outlet conduit 110 via outlet port 120 on lid 125.

Valve assembly 500 may include a first (outer) enclosure 530 and a second (inner) enclosure 535 that is disposed inside first (outer) enclosure 530. Inner enclosure 535 may include an exterior surface 510 and an interior surface 520 that is opposite of exterior surface 510. Interior surface 520 may at least partially define cavity 135. Exterior surface 510 may be disposed in an outwards direction from inner enclosure 535, such that exterior surface 510 faces radially outwards towards outer enclosure 530. Interior surface 520 may be disposed in an inwards direction from inner enclosure 535, such that interior surface 520 faces radially inwards towards cavity 135. In some embodiments, inner enclosure 535 may be movably coupled to lid 125 and/or outer enclosure 530. Inner enclosure 535, and particularly one or more of exterior surface 510 and interior surface 520, may be magnetic. For example, one or more of surfaces 510, 520 of inner enclosure 535 may be formed of a magnetic material, layered with a magnetic coating, and/or coupled to a magnetic film to provide magnetic properties along the corresponding surfaces on inner enclosure 535.

Still referring to FIG. 8, inner enclosure 535 may be configured to move (e.g. rotate) independently from and relative to outer enclosure 530. In the example, outer enclosure 530 may be securely fixed relative to lid 125 and inner enclosure 535. Inner enclosure 535 may include an agitation mechanism 540 disposed within cavity 135. Agitation mechanism 540 may include various suitable sizes, shapes, and/or cross-sectional configurations. For example, agitation mechanism 540 may include a ball, a pin, a piston, a body, or other mechanism that is sized, shaped, and/or otherwise configured to agitate powdered agent 10. In the example, agitation mechanism 540 may include a ball that is magnetic and has a spherical shape.

Inner enclosure 535 may be configured to rotate relative to outer enclosure 530 to manipulate and/or move agitation mechanism 540 within cavity 135. Stated differently, at least a portion of valve assembly 500 (e.g., inner enclosure 535) may be configured to rotate, thereby causing agitation mechanism 540 to move within cavity 135 to agitate powdered agent 10. In the example, inner enclosure 535 may be configured to generate a magnetic field about cavity 135, such as prior to or upon movement (e.g., rotation) relative to outer enclosure 530. For example, the magnetic field generated by inner enclosure 535 may be configured to urge agitation mechanism 540 to move within cavity 135 due to the magnetic field generated by inner enclosure 535 moving simultaneously with the corresponding movement (e.g. rotation) of inner enclosure 535 within outer enclosure 530.

Still referring to FIG. 8, agitation mechanism 540 may be configured to agitate and/or mix powdered agent 10 and pressurized fluid within cavity 135 in response to agitation mechanism 540 moving inside inner enclosure 535 to prevent and/or minimize clogging of outlet conduit 110 and/or catheter 36 (see FIG. 1) during delivery of powdered agent 10.

Although valve assemblies 100, 200, 300, 400, 500 are shown and described herein as being implemented in delivery system 10, and particularly in a handheld device, it should be appreciated that valve assemblies 100, 200, 300, 400, 500 may be configured and operable in various other suitable configurations. For example, valve assemblies 100, 200, 300, 400, 500 may be directly coupled to a delivery device (e.g., an endoscope, a catheter, etc.) that is positioned adjacent to a target treatment site within a subject (e.g., a patient).

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description. The features described herein may be used alone or in combination and are not mutually exclusive.

What is claimed is:

1. A device for delivering an agent, comprising:
a valve assembly including a cavity for storing the agent and at least one agitation mechanism movably disposed inside the cavity;
an inlet conduit fluidly coupled to a fluid source and the valve assembly; and
an outlet conduit fluidly coupled to the valve assembly, wherein the inlet conduit and the outlet conduit are each in fluid communication with the cavity;
wherein the at least one agitation mechanism is configured to agitate the agent within the cavity in response to the at least one agitation mechanism moving inside the cavity; and
wherein the valve assembly is configured to:
urge the at least one agitation mechanism to move inside the cavity in response to receiving a fluid from the fluid source via the inlet conduit;
combine the fluid with the agent within the cavity in response to the at least one agitation mechanism moving inside the cavity; and
deliver a combination of the agent and the fluid out of the cavity through the outlet conduit.

2. The device of claim 1, wherein the fluid source includes a pressurized canister, and the fluid includes a pressurized gas.

3. The device of claim 1, wherein the cavity includes at least one measured dose of the agent.

4. The device of claim 1, wherein, when combining the agent with the fluid within the cavity, the valve assembly is configured to direct the fluid towards the at least one agitation mechanism, thereby moving the at least one agitation mechanism within the cavity and agitating the agent.

5. The device of claim 1, wherein the valve assembly includes an outlet port coupled to the outlet conduit and a mesh coupled to the outlet port;
wherein the mesh is configured to inhibit at least a portion of the agent from exiting the cavity via the outlet port, and the outlet port includes a funnel shape configured to guide the combination of the agent and the fluid towards the outlet conduit.

6. The device of claim 1, wherein the at least one agitation mechanism includes a ball that is configured to generate turbulence within the cavity to combine the fluid and the agent in response to the fluid from the inlet conduit urging the at least one agitation mechanism to move within the cavity.

7. The device of claim 1, wherein the valve assembly includes:
one or more interior surfaces defining the cavity; and
a wire extending along at least one of the one or more interior surfaces, wherein the wire is configured to generate a magnetic pulse and the at least one agitation mechanism is magnetic; and
wherein the wire is configured to magnetically couple with the at least one agitation mechanism when generating the magnetic pulse, thereby causing the at least one agitation mechanism to move within the cavity and relative to the one or more interior surfaces to agitate the agent.

8. The device of claim 1, wherein the valve assembly includes:
one or more interior surfaces defining the cavity; and
a membrane positioned adjacent to at least one of the one or more interior surfaces;
wherein the membrane is configured to agitate the agent with the fluid received within the cavity in response to the membrane vibrating against the at least one of the one or more interior surfaces.

9. The device of claim 1, wherein the valve assembly includes a base including at least one magnetic mechanism disposed adjacent to the cavity, and the at least one agitation mechanism is magnetic;
wherein the base is configured to move relative to the cavity and the at least one magnetic mechanism is configured to magnetically couple with the at least one agitation mechanism, thereby causing the at least one agitation mechanism to move within the cavity and agitate the agent.

10. The device of claim 9, wherein the at least one agitation mechanism is configured to create a vortex within the cavity in response to the at least one agitation mechanism moving therein, thereby agitating the agent with the fluid.

11. The device of claim 9, wherein the base includes a central body and a plurality of blades coupled to the central body, at least one of the plurality of blades includes the at least one magnetic mechanism;
wherein the central body is configured to rotate the plurality of blades relative to the cavity, thereby causing the at least one magnetic mechanism to rotate the at least one agitation mechanism.

12. The device of claim 11, wherein the at least one magnetic mechanism is configured to generate a magnetic field that moves the at least one agitation mechanism within the cavity.

13. The device of claim 1, wherein the valve assembly includes an outer enclosure and an inner enclosure disposed inside the outer enclosure, wherein the inner enclosure includes one or more interior surfaces defining the cavity; and
wherein the inner enclosure is configured to move relative to the outer enclosure, thereby causing the at least one agitation mechanism to move within the cavity to agitate the agent with the fluid.

14. The device of claim 13, wherein the one or more interior surfaces are magnetic and the at least one agitation mechanism is magnetic, such that the one or more interior surfaces are configured to magnetically couple with the at least one agitation mechanism.

15. The device of claim 14, wherein the one or more interior surfaces are configured to generate a magnetic field, thereby causing the at least one agitation mechanism to move within the cavity to agitate the agent in response to movement of the inner enclosure relative to the outer enclosure.

16. A device for delivering an agent, comprising:
a valve assembly including a cavity and an agitation mechanism movably disposed inside the cavity; and
a base including a magnetic mechanism movably disposed outside the cavity, wherein the magnetic mechanism is magnetically coupled to the agitation mechanism, and the base is configured to move relative to the valve assembly, thereby causing movement of the magnetic mechanism;
wherein the agitation mechanism is configured to move within the cavity to agitate the agent stored therein in response to movement of the magnetic mechanism outside the cavity; and
wherein the valve assembly is configured to mix the agent with a fluid received within the cavity and deliver a mixture of the agent and the fluid out of the cavity in response to movement of the agitation mechanism in the cavity.

17. The device of claim 16, wherein the agitation mechanism is magnetic and the magnetic mechanism is configured to urge the agitation mechanism to move within the cavity in response to a corresponding movement of the base outside the cavity.

18. The device of claim 16, wherein the base includes a central body and a plurality of blades extending outwardly from the central body, wherein the magnetic mechanism is coupled to at least one of the plurality of blades.

19. The device of claim 18, wherein the plurality of blades is configured to rotate relative to the valve assembly, thereby rotating the magnetic mechanism relative to the cavity.

20. A device for delivering an agent, comprising:
a valve assembly including an outer enclosure, an inner enclosure including a cavity storing the agent, and an agitation mechanism, the inner enclosure is movably disposed inside the outer enclosure and the agitation mechanism is movably disposed inside the cavity;
wherein the inner enclosure is magnetically coupled to the agitation mechanism, such that the inner enclosure is configured to move the agitation mechanism within the cavity in response to moving relative to the outer enclosure; and
wherein the agitation mechanism is configured to agitate the agent stored inside the cavity in response to the inner enclosure moving within the outer enclosure.
